(19) United States
(12) Patent Application Publication
(10) Pub. No.: US 2024/0120082 A1
(43) Pub. Date: Apr. 11, 2024

(54) APPARATUS AND METHODS OF PREDICTING FAULTS IN DIAGNOSTIC LABORATORY SYSTEMS

(71) Applicant: Siemens Healthcare Diagnostics Inc., Tarrytown, NY (US)

(72) Inventors: Vivek Singh, Princeton, NJ (US); Rayal Raj Prasad Nalam Venkat, Princeton, NJ (US); Yao-Jen Chang, Princeton, NJ (US); Venkatesh NarasimhaMurthty, Hillsborough, NJ (US); Benjamin S. Pollack, Jersey City, NJ (US); Ankur Kapoor, Plainsboro, NJ (US)

(73) Assignee: Siemens Healthcare Diagnostics Inc., Tarrytown, NY (US)

(21) Appl. No.: 18/264,685

(22) PCT Filed: Feb. 7, 2022
(86) PCT No.: PCT/US2022/070546

§ 371 Date: Aug. 8, 2023

Related U.S. Application Data
(60) Provisional application No. 63/147,155, filed on Feb. 8, 2021.

(51) Int. Cl. G16H 40/40; B01L 99/00; G16H 10/40

(52) U.S. Cl. CPC G16H 40/40; B01L 99/00; G16H 10/40; B01L 2200/143; B01L 2300/0627

(57) ABSTRACT

Methods of predicting a fault in a diagnostic laboratory system include providing one or more sensors; generating data using the one or more sensors; inputting the data into an artificial intelligence algorithm, the artificial intelligence algorithm configured to predict at least one fault in the diagnostic laboratory system in response to the data; and predicting at least one fault in the diagnostic laboratory system using the artificial intelligence algorithm. Other methods, systems, and apparatus are also disclosed.

### CROSS REFERENCE TO RELATED APPLICATION

This application claims the benefit of U.S. Provisional Patent Application No. 63/147,155, entitled “APPARATUS AND METHODS OF PREDICTING FAULTS IN DIAGNOSTIC LABORATORY SYSTEMS” filed Feb. 8, 2021, the disclosure of which is hereby incorporated by reference in its entirety for all purposes.

### FIELD

Embodiments of the present disclosure relate to apparatus and methods of predicting faults in diagnostic laboratory systems.

### BACKGROUND

Diagnostic laboratory systems may conduct chemical analysis or tests on biological specimens that may be contained in specimen containers. The diagnostic laboratory systems may include a plurality of instruments and individual modules. Each of the instruments may include a plurality of modules and may perform one or more processes and/or analysis on the specimen containers and/or the specimens. Some of the modules and instruments may include components therein that process the specimen containers or analyze the specimens. In some embodiments, a first module may perform a process, such as centrifuging, that prepares a specimen for analysis in a second module. The second module may include one or more components that perform the analysis on the specimen.

Should a module or a component within a module experience a fault (e.g., a malfunction), the ability of the diagnostic laboratory system to perform analysis may be severely reduced. For example, if a component in the first module malfunctions, the analyzing capability may be reduced. The malfunction or reduced analyzing capability of a module may reduce the testing capacity of the entire diagnostic laboratory system.

Thus, improved methods and apparatus of predicting faults in instruments, modules, and/or components thereof in diagnostic laboratory systems are sought.

### SUMMARY

According to a first aspect, a method of predicting a fault in a diagnostic laboratory system is provided. The method includes providing one or more sensors; generating data using the one or more sensors; inputting the data into an artificial intelligence algorithm, the artificial intelligence algorithm configured to predict at least one fault in the diagnostic laboratory system in response to the data; and predicting at least one fault in the diagnostic laboratory system using the artificial intelligence algorithm.

In a further aspect, a method of predicting a fault in a component of a module in a diagnostic laboratory system is provided. The method includes providing one or more sensors in the module of the diagnostic laboratory system; generating data using the one or more sensors; inputting the data into an artificial intelligence algorithm, the artificial intelligence algorithm configured to predict a fault of the component in response to the data; and predicting a probability of a fault in the component using the artificial intelligence algorithm.

In another aspect, a diagnostic laboratory system is provided. The diagnostic laboratory system includes one or more sensors configured to generate data; and a computer configured to execute an artificial intelligence algorithm, the artificial intelligence algorithm configured to: receive the data; and predict at least one fault in a component of the diagnostic laboratory system in response to the data.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. This disclosure is intended to cover all modifications, equivalents, and alternatives falling within the scope of the claims and their equivalents.

### BRIEF DESCRIPTION OF THE DRAWINGS

The drawings, described below, are for illustrative purposes, and are not necessarily drawn to scale. Accordingly, the drawings and descriptions are to be regarded as illustrative in nature, and not as restrictive. The drawings are not intended to limit the scope of the disclosure in any way.

FIG. 1 illustrates a block diagram of a diagnostic laboratory system including a plurality of modules and instruments according to one or more embodiments.

FIG. 2A illustrates a side elevation view of a specimen container located in a carrier, wherein a specimen is located in the specimen container according to one or more embodiments.

FIG. 2B illustrates a side elevation view of a specimen container located in a carrier, wherein a specimen, having undergone a centrifuging process, is located in the specimen container according to one or more embodiments.

FIG. 3 illustrates a block diagram of an instrument of a diagnostic laboratory system, wherein the instrument includes a plurality of modules according to one or more embodiments.

FIG. 4 illustrates a top plan view of an embodiment of a quality check module included in an embodiment of a diagnostic laboratory system according to one or more embodiments.

FIG. 5 illustrates a side, partially cross-sectioned view of an aspiration and dispensing module according to one or more embodiments.

FIG. 6 is a graph illustrating traces of pressure (Pa) of a pipette assembly aspirating a specimen showing a functioning aspiration system and a faulty aspiration system according to one or more embodiments.

FIG. 7 illustrates a block diagram depicting an embodiment of an analyzer module of a diagnostic laboratory system according to one or more embodiments.

FIG. 8 illustrates a flowchart depicting a method of predicting a fault in a diagnostic laboratory system according to one or more embodiments.

FIG. 9 illustrates a flowchart depicting a method of predicting a fault in a component of a module in a diagnostic laboratory system according to one or more embodiments.

### DETAILED DESCRIPTION

Diagnostic laboratory systems analyze (e.g., test) specimens from patients to determine the presence and/or concentration of one or more analytes or constituents within the specimens provided. For example, doctors or other medical providers diagnosing patients may order analyses of biological specimens (e.g., specimens) taken from patients. The specimens are typically collected in specimen containers and sent to a diagnostic laboratory system along with the test orders generated by the medical professionals.

The diagnostic laboratory system may include one or more modules that may process specimens and/or specimen containers to prepare the specimen containers and/or the specimens for analysis (e.g., testing). One or more other modules may perform the analyses on the specimens. In some embodiments, a first module may prepare a specimen or specimen container for analysis by a second module. For example, a first module may analyze specimens to determine whether the specimens are in condition for analysis by a second module. In another example, a first module may read barcodes on specimen containers or perform centrifuging, and/or aliquoting on specimens. The second module may perform the analyses on the specimens.

Some modules that process specimen containers may identify the specimens contained therein, for example. Such identification may include reading indicia, such as barcodes, attached to the specimen containers. Barcode readers and/or other imaging devices may read the barcodes, which may be used to correlate sample identifications with a laboratory information system (LIS) to provide information on specific analyses that are to be performed on the specimens. Some modules may include imaging devices that capture images of the specimen containers to identify the shapes and/or cap types of the specimen containers to identify the specific type of specimen container containing a specimen. Other modules may perform other processing of the specimen containers, such as removing caps (decapping) and other processes.

Analyzer modules (analyzers) may perform one or more analyses or tests, such as assays or clinical chemistry analyses, on the specimens. In some embodiments, reagents and the like may be added to the specimens to determine the presence of and/or concentrations of certain analytes. The analyzers may also determine the presence and/or concentrations of other substances, such as certain antigens, proteins, or drugs. In some embodiments, vision systems may be used to determine light absorption at different frequencies and/or fluorescence emissions of the specimens with and without reagents.

Some diagnostic laboratory systems may include one or more instruments that may contain several modules within them, wherein each module may perform a plurality of processes and/or analyses on specimens and specimen containers.

Some diagnostic laboratory systems may be one hundred meters long and/or wide, for example, and may perform thousands or even tens of thousands of analyses (e.g., tests) daily. These diagnostic laboratory systems may include hundreds of modules and/or instruments with many different types of modules that have to be maintained and calibrated in order for the diagnostic laboratory systems to provide accurate analyses of specimens and/or prescreening of specimens and/or specimen containers. When a single module of the diagnostic laboratory system experiences a fault, or is deactivated for calibration or maintenance, the efficiency of the diagnostic laboratory system can decrease, and the diagnostic laboratory system may not be able to perform as many analyses as when the diagnostic laboratory system is operating with all the modules functioning as intended.

Technicians maintaining and/or calibrating conventional diagnostic laboratory systems rely on preventive maintenance schedules to periodically update or replace the modules, instruments, and/or components in the modules and instruments. Thus, certain modules, instruments, and/or components thereof may be maintained based on a periodic schedule and not on the actual condition of the modules, instruments, and/or components. In some embodiments, modules, instruments and/or the components may be replaced based on their estimated lifetime. However, if a module, instrument, or component thereof experiences a fault (e.g., malfunctions) prior to its expected lifetime, malfunction of the diagnostic laboratory system and/or portions thereof may occur. The malfunction may last until a technician visits the site of the diagnostic laboratory system to troubleshoot the diagnostic laboratory system and resolve the system fault.

When preventive maintenance schedules are relied upon, modules, instruments, and components thereof may be replaced to reduce the risk of device failure even if the modules, instruments, and components thereof are operating entirely properly. Replacing modules, instruments, and components that are otherwise functioning properly unnecessarily increases the costs and efficiency of operating diagnostic laboratory system.

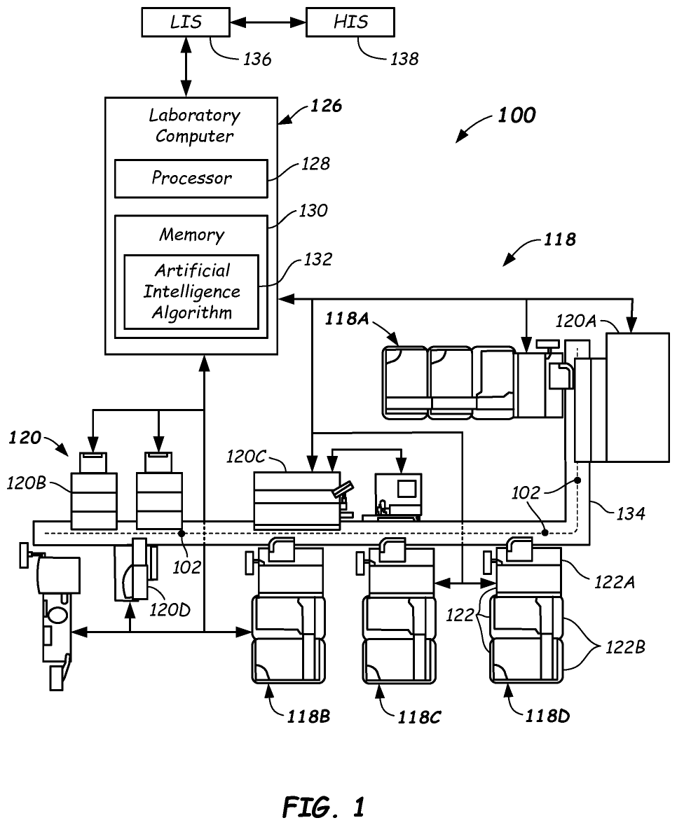

One or more of the modules and/or instruments may perform self-tests and/or monitor sensors within the modules and instruments. Apparatus and methods described herein include artificial intelligence algorithms (e.g., neural networks) implementing trained models to analyze the internal tests, the sensor data, and/or analyses results performed on the specimens to predict faults of modules and/or instruments. The artificial intelligence algorithm, based on the various inputs, predicts which modules, instruments, and/or components are likely to experience faults. In some embodiments, the artificial intelligence algorithms may predict when certain modules, instruments, and/or components will malfunction. These and other methods, systems, and apparatus are described in greater detail with reference to FIGS. 1-9 herein.

Reference is now made to FIG. 1, which illustrates a block diagram of an embodiment of a diagnostic laboratory system 100 that performs analyses (e.g., tests or assays) or other processes on biological specimens (specimens). The specimens may include whole blood, blood serum, blood plasma, urine, cerebrospinal fluids, or other bodily fluids taken from patients. The specimens are collected from the patients and stored in specimen containers 102 (a few labelled), which are illustrated as being transported through the diagnostic laboratory system 100 on track 134. Track 134 may be any suitable track capable of moving the specimen containers 102. Test orders, which may include analyses that are to be performed on specific specimens, may also be received in the diagnostic laboratory system 100, such as from a laboratory information system (LIS) 136. The test orders may be processed to instruct specific instruments and modules to perform certain processes and/or analyses as described herein.

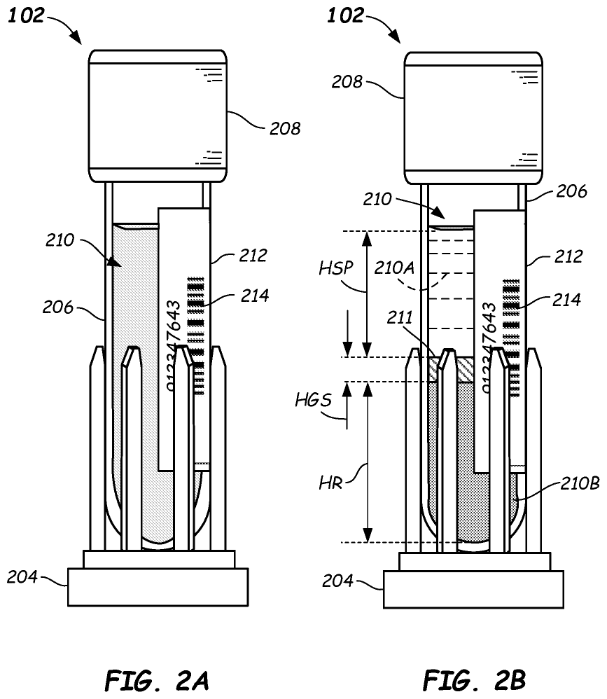

Reference is now made to FIG. 2A, which illustrates a side elevation view of an embodiment of a specimen container 102 in a carrier 204. The specimen container 102 may be identical or substantially similar to the specimen containers 102 (FIG. 1). The specimen container 102 includes a tube 206 and may be capped by a cap 208. A specimen 210 may be located in the tube 206. The specimen 210 may be any liquid (biological liquid) that is to be analyzed by the diagnostic laboratory system 100 (FIG. 1). In the embodiment of FIG. 2A, the specimen 210 has not been subjected to a centrifuge process.

The tube 206 may have a label 212 attached thereto, and the label 212 may contain information, such as a barcode 214 or other identifier, related to the specimen 210. In some embodiments, the label 212 may include numbers and/or letters that identify the specimen 210. Images of the barcode 214 may be captured by one or more of the imaging devices located within the diagnostic laboratory system 100. In some embodiments, images of the specimen 210 also may be captured by the imaging devices as described herein. In other embodiments, images of the cap 208 may be captured by the imaging devices. In some embodiments, images of the tube 206, the cap 208, and/or the specimen 210 may be captured by one or more imaging devices.

Additional reference is made to FIG. 2B, which is a side elevation view of an embodiment of the specimen container 102 wherein the specimen 210 has undergone a centrifuge process to separate components of the specimen 210. For example, the embodiment of the specimen 210 shown in FIG. 2A may have undergoing centrifuging by one of the modules 120 in the diagnostic laboratory analyzer 100, for example. Heavier components in the specimen 210 may settle toward the bottom of the tube 206 and lighter components may settle toward the top of the tube 206 in response to the centrifuge process. In the embodiment of FIG. 2B, the specimen 210 may be a blood sample. During the centrifuge process, serum or plasma 210A may be separated from red blood cell portion 210B. A separator 211 (e.g., a gel separator) may separate the serum or plasma 210A from the red blood cell portion 210B.

The serum or plasma 210A is illustrated as having a height HSP, the separator 211 is illustrated as having a height HGS, and the red blood cell portion 210B is illustrated as having a height HR. In some embodiments, the serum or plasma 210A is analyzed, which involves aspirating at least some of the serum or plasma 210A. To aspirate correctly, the height HSP of the serum or plasma 210A may be measured and used during aspiration processes. For example, the height HSP may enable a processor or the like to determine the volume of the serum or plasma 210A in the specimen container 102. The height HSP may be used to provide information to other modules as to the depth that an aspiration probe may need to extend into the specimen container 102 to enable aspiration of the serum or plasma 210A and/or the available amount of the serum or plasma 210A.

Referring again to FIG. 1, the diagnostic laboratory system 100 may include a plurality of instruments 118 and modules 120 that may process and/or prepare the specimen containers 102 for analyses and may perform analyses on the specimens located therein.

In the embodiment of FIG. 1, the diagnostic laboratory system 100 includes four instruments 118, which are referred to individually as a first instrument 118A, a second instrument 118B, a third instrument 118C, and a fourth instrument 118D. In the embodiment of FIG. 1, the diagnostic laboratory system 100 may include a plurality of modules 120, some of which are labeled as a first module 120A, a second module 120B, a third module 120C, and a fourth module 120D. More or fewer modules may be present.

The instruments 118 may each include two or more modules, wherein some of the modules may perform functions identical to or similar to functions performed by one or more of the modules 120. Reference is made to the fourth instrument 118D, which may be similar or identical to the other instruments. The fourth instrument 118D may include three modules 122, which may include a processing module 122A and one or more analyzer modules 122B. The processing module may prepare specimens for analysis (e.g., testing) and may identify specimen containers 102 received in the fourth instrument 122D as described further below. The analyzer modules 122B may perform analyses on the specimens as described further below.

The diagnostic laboratory system 100 may include a laboratory computer 126 that may be in communication with the instruments 118 and the modules 120 and LIS 136. The laboratory computer 126 may be proximate or remote from the instruments 118 and the modules 120. The laboratory computer 126 may include a processor 128 and memory 130, wherein the processor 128 executes programs that may be stored in the memory 130. One of the programs stored in the memory 130 may be at least one artificial intelligence algorithm 132 (AI algorithm 132). In some embodiments, the AI algorithm 132 described herein may be stored in the memory 130 or in another computer, such as a computer (not shown) that is remote from the diagnostic laboratory system 100.

As described herein, the AI algorithm 132 receives data, such as sensor and/or analyses data, from the instruments 118 and/or the modules 120 and, in response to the data, predicts when one or more of the instruments 118 and/or modules 120 (or components thereof) of the diagnostic laboratory system 100 may experience a fault as described further below. For example, the AI algorithm 132 may predict a probability that a component in a module of the diagnostic laboratory system 100 will experience a fault within a predetermined period of time.

The laboratory information system (LIS) 136 may be coupled to the laboratory computer 126 and a hospital information system (HIS) 138 may be coupled to the LIS 136. A medical professional or the like may enter test orders into the HIS 138. The test orders indicate the types of analyses (e.g., tests) are to be conducted on specific specimens. The specimens can be collected into specimen containers 102 and sent to the diagnostic laboratory system 100. The LIS 136 or other logic at an input/output device (I/O device) coupled to the track 134 may then schedule the analyses so that the analyses and related processes are performed on specific instruments 118 and/or modules 120. In some embodiments, the LIS 136 may be implemented in the laboratory computer 126.

The track 134 can be configured to transport the specimen containers 102 throughout the diagnostic laboratory system 100. For example, the track 134 may transport the specimen containers 102 to specific ones of the instruments 118 and the modules 120. In some embodiments, the carriers 204 (FIGS. 2A-2B) may be self-propelled and the track 134 may provide a mechanism on which the carriers 204 move the specimen containers 102. Specimen containers 102 may move in multiple directions along the track 134.

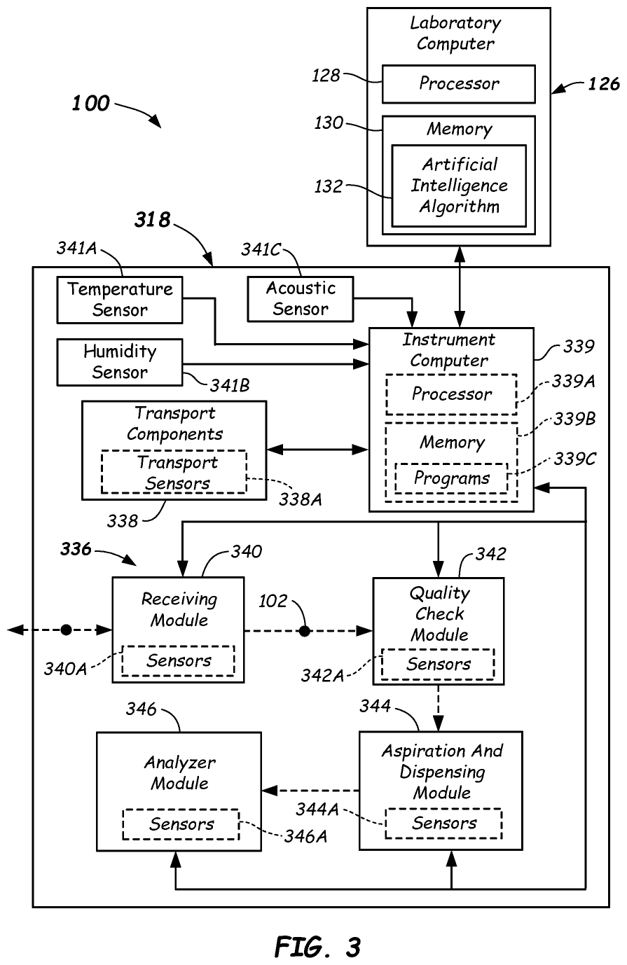

Additional reference is made to FIG. 3, which illustrates a block diagram of an example embodiment of an instrument 318. The instrument 318 may be identical or substantially similar to one or more of the instruments 118 (FIG. 1). In the embodiment of FIG. 3, the instrument 318 is configured to perform one or more analyses on specimens, such as the specimen 210 (FIGS. 2A-2B). The instrument 318 may be further configured to perform a process on the specimen containers 102. The instrument 318 may include other modules and instruments than those shown in FIG. 3.

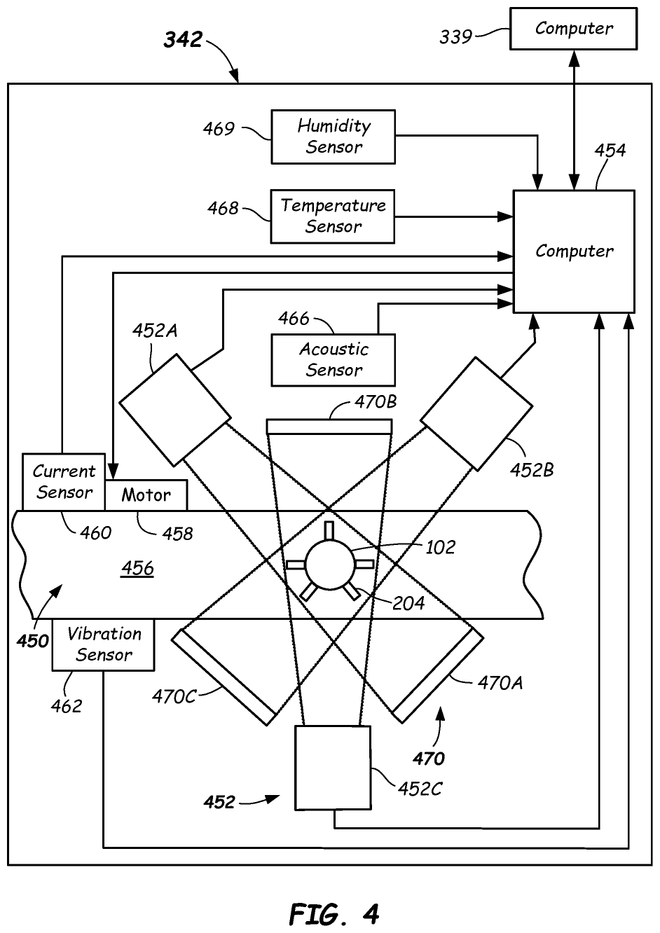

The instrument 318 may be configured to receive specimen containers 102 and may transport the specimen containers 102 and/or specimens (e.g., specimen 210—FIGS. 2A-2B) throughout the instrument 318. The instrument 318 may include one or more modules 336 that prepare specimens for analysis and that perform one or more analyses on the specimens. The instrument 318 may also include transport components 338 that are configured to transport specimen containers, reagents, specimens, and/or other items throughout the instrument 318, such as between different ones of the modules 336 in the instrument 318. The transport components 338 may include one or more conveyor devices and/or one or more robots, examples of which are shown at least in FIG. 4.

The instrument 318 may include a temperature sensor 341A configured to measure temperature and generate data indicative of temperature. The temperature sensor 341A may measure ambient air temperature and/or the temperature of one or more components within the instrument 318. If the instrument is operated at high temperature, one or more of the components or modules 336 may experience premature or imminent faults that may be detected by the AI algorithm 132 as described herein. The instrument 318 also may include a humidity sensor 341B configured to measure humidity and generate data indicative of ambient humidity. If the instrument 318 operates in relatively high or relatively low ambient humidity, one or more components or modules 336 may experience premature or imminent faults that may be detected by the AI algorithm 132. The instrument 318 also may include an acoustic sensor 341C configured to measure sound of one or more components or of the instrument 318 and generate data indicative of sound within the instrument 318. If the instrument 318 generates excessive noise, one or more components or modules 336 may experience premature or imminent faults that may be detected by the AI algorithm 132. The data generated by the temperature sensor 341A, the humidity sensor 341B, and/or the acoustic sensor 341C also may be used to train the AI algorithm 132 as described herein.

The transport components 338 may include and/or be associated with transport sensors 338A that sense one or more parameters associated with the transport components 338. In some embodiments, the transport sensors 338A may include electric current sensors that are configured to measure electric current drawn through one or more motors (not shown in FIG. 3) that drive the transport components 338 and output data indicative of the electric current drawn. In some embodiments, the transport sensors 338A may include an acoustic sensor configured to measure noise and/or vibration and generate data indicative of the noise and/or vibration. Excessive noise and/or vibration may be indicative of one or more of the transport components 338 experiencing a fault or indicative of an impending fault. The data generated by the transport sensors 338A may be input to the AI algorithm 132 and used to predict a fault in the instrument 318 and/or the diagnostic laboratory system 100. In some embodiments, the data generated by the transport sensors 338A may be used to train the AI algorithm 132.

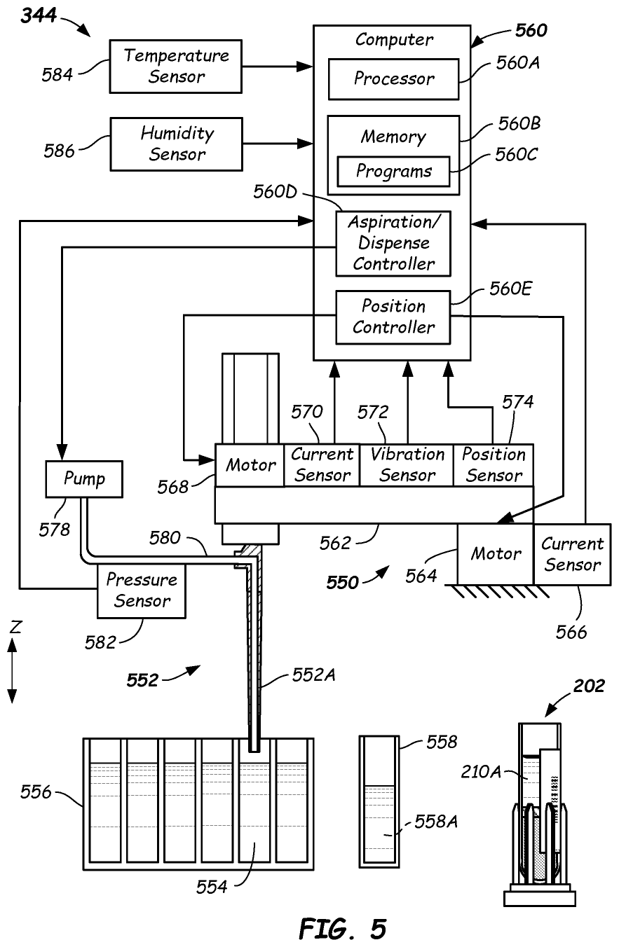

The transport sensors 338A may also include position sensors configured to determine locations of objects and components in the instrument 318. For example, positions of the specimen containers 102 and/or aliquots within the instrument 318 may be sensed (e.g., measured). The transport sensors 338A may also measure the positions of one or more robots (e.g., robot 550—FIG. 5) or components thereof as described herein.

The instrument 318 may include an instrument computer 339 that may send instructions to and receive data from the modules 336 and other components, such as the transport components 338, the transport sensors 338A, and other sensors. The computer 339 may include a processor 339A and memory 339B that may store one or more programs 339C. One or more of the programs 339C may instruct the transport components 338 and/or the modules 336 to perform predetermined processes, such as preparing the specimens for testing and running the analyses on the specimens. The instrument computer 339 may be in communication with the laboratory computer 126 (FIG. 1). In other embodiments, the laboratory computer 126 and the instrument computer 339 may be implemented in a single computer. Thus, the memory 339B and the memory 130 may be implemented as a single memory.

In some embodiments, the instrument 318 may include a receiving module 340 that receives specimen containers 102, such as from the track 134 (FIG. 1) and may return the specimen containers 102 to the track 134. In some embodiments, the receiving module 340 may receive (e.g., aspirate) the specimen (e.g., the specimen 210—FIGS. 2A-2B), such as a serum or plasma (e.g., the serum or plasma 210A—FIG. 2B) in the specimen. The receiving module 340 may be configured similar or identical to the processing module 122A (FIG. 1). The receiving module 340 also may be similar to or identical to one or more of the modules 120 (FIG. 1). The receiving module 340 may include motors, robots, gates, conveyors, and the like that are configured to receive and/or return the specimen containers 102.

The receiving module 340 may also include components configured to process the specimen containers 102 and or specimens located therein. In some embodiments, the components may remove and/or replace caps (e.g., cap 208—FIGS. 2A-2B) on the specimen containers 102. In some embodiments, the components may perform centrifuging on the specimens located in the specimen containers 102 to separate liquids in the specimens. For example, the specimens may be separated into a serum or plasma (e.g., serum or plasma 210A—FIG. 2B) and a clot (e.g., clot 210B—FIG. 2B). The components may perform other functions.

The receiving module 340 may include and/or be associated with sensors 340A that monitor components within the receiving module 340. For example, the sensors 340A may determine whether centrifuges, capping and decapping devices, and the like are operating correctly. The sensors 340A may also include imaging devices and the like that read labels (e.g., label 212—FIGS. 2A-2B) on the specimen containers 102. The sensors 340A may also include sensors, such as optical and magnetic sensors that determine the positions of specimen containers 102 within or proximate the receiving module 340. Data generated by the sensors 340A may be input to the AI algorithm 132 (FIG. 1) to predict one or more faults in the instrument 318 and/or the diagnostic laboratory system 100 (FIG. 1). The data also may be input to the AI algorithm 132 to train the AI algorithm 132.

In the embodiment of FIG. 3, the instrument 318 may include a quality check module 342. The quality check module 342 may capture images of the specimens and/or the specimen containers 102. In some embodiments, image of the specimens and/or specimen containers are captured to determine the quality of the specimens and/or the specimen containers 102 as described herein. Additional reference is made to FIG. 4, which illustrates a top plan view of an embodiment of the quality check module 342. The quality check module 342 is one of only a plurality of different embodiments of imaging-type modules that may be used in an instrument or a module of the diagnostic laboratory system 100. The sensors and other devices described with reference to the quality check module 342 may be implemented in other modules in the instrument 318 or modules 120 of the diagnostic laboratory system 100.

The quality check module 342 may include a transport system 450 that is configured to transport the specimen container 102 (FIGS. 2A-2B), and/or the carrier 204 (FIGS. 2A-2B) through the quality check module 342. One or more imaging devices 452 may capture one or more images of the specimen container 102 when the specimen container 102 is located within the quality check module 342. In the embodiment of FIG. 4, the quality check module 342 includes three imaging devices 452, which are referred to individually as a first imaging device 452A, a second imaging device 452B, and a third imaging device 452C. The quality check module 342 may include more or fewer imaging device 452. The quality check module 342 may also include a computer 454 that is in communication with the components of the quality check module 342. The computer 454 may operate the components, receive data generated by the components, and/or analyze the data. In some embodiments, the computer 454 may be in communication with the computer 339 (FIG. 3) and/or the laboratory computer 126 (FIG. 3).

The transport system 450 may be configured to transport the specimen container 102 into and out of the quality check module 342. The transport system 450 may also be configured to stop the specimen container 102 at an imaging location within the quality check module 342. An imaging location is a location in the quality check module 342 where one or more of the imaging devices 452 may capture an image of the specimen container 102 and/or the specimen located therein. The transport system 450 may include a conveyor 456 that is operated by a motor 458. The conveyor 456 may be any device that facilitates the movement of the specimen container 102 within the quality check module 342. The motor 458 may be controlled by instructions generated by the computer 454.

A current sensor 460 may be configured to measure electric current drawn by the motor 458 and may output data indicative of the measured current. The measured current may be output to the computer 454 where the measured current may be analyzed and/or output to the computer 339 and/or the laboratory computer 126 (FIG. 2). The measured current may be input to the AI algorithm 132. Changes in current or current greater than or less than a predetermined current value may be indicative of an impending fault of the motor 458 or other components in the transport system 450. For example, the motor 458 may have internal problems that cause a drag on the motor 458, which may cause the motor 458 to draw excessive current. The excessive current may also be indicative of drag on the conveyor 456, wherein the motor 458 draws excessive current to overcome the drag on the conveyor 456. The data generated by the current sensor 460 may be used to train the artificial intelligence algorithm 132 (FIG. 1). In some embodiments, current may be sensed from other components within the quality check module 342 and used in the manner described with reference to the current sensor 460. In some embodiments, the measured current may be used with other data to predict impending faults in the quality check module 342 and/or other components in the diagnostic laboratory system 100.

In some embodiments, the quality check module 342 may include a vibration sensor 462 that is configured to measure vibration in one or more components within the quality check module 342. The vibration sensor 462 may generate vibration data that is transmitted to the computer 454, the computer 339, and/or the laboratory computer 126 (FIG. 1). The vibration data may be input to the AI algorithm 132 to predict impending faults and/or to train the artificial intelligence algorithm. Excessive vibration may be indicative of an imminent fault in the quality check module 342. For example, wearing and/or loose components may vibrate prior to failure.

In some embodiments, the quality check module 342 may also include an acoustic sensor 466 configured to measure sound (e.g., noise) in one or more components within the quality check module 342. The acoustic sensor 466 may generate noise or sound data that is transmitted to the computer 454, the computer 339, and/or the laboratory computer 126 (FIG. 1). The noise data may be input to the AI algorithm 132 to predict a fault and/or to train the AI algorithm 132. Excessive noise may be indicative of an imminent fault in the quality check module 342. For example, wearing and/or loose components may generate excessive noise before they experience faults.

In some embodiments, the quality check module 342 may also include a temperature sensor 468 that measures temperature in one or more components within the quality check module 342. The temperature sensor 468 may also measure ambient air temperature within the quality check module 342. The temperature sensor 468 may generate temperature data that is transmitted to the computer 454, the computer 339, and/or the laboratory computer 126 (FIG. 1). The temperature data may be input to the AI algorithm 132. Excessive temperature or low temperature may be used by the AI algorithm 132 to predict an imminent fault in the quality check module 342 or other components in the diagnostic laboratory system 100 (FIG. 1). For example, components on the verge of experiencing a fault may operate at high or low temperatures. In addition, when the quality check module 342 is operated at high temperatures, components therein may experience premature faults. The temperature also may be used to train the AI algorithm 132.

In some embodiments, the quality check module 342 may also include a humidity sensor 469 configured to measure ambient air humidity within the quality check module 342. The humidity sensor 469 may generate humidity data that is transmitted to the computer 454, the computer 339, and/or the laboratory computer 126 (FIG. 1). The humidity data may be input to the AI algorithm 132. Excessive humidity or low humidity may be used by the AI algorithm 132 to predict an imminent fault in the quality check module 342 or other components in the diagnostic laboratory system 100 (FIG. 1). For example, when the quality check module 342 is operated in high humidity, components therein may experience premature faults. The humidity data also may be used to train the AI algorithm 132.

In some embodiments, the quality check module 342 may include one or more illumination sources 470 that are configured to illuminate the specimen container 102 when at an imaging location. In the embodiment of FIG. 4, the quality check module 342 may include three illumination sources 470, which are referred to individually as a first illumination source 470A, a second illumination source 470B, and a third illumination source 470C. The illumination sources 470 may be turned off and on by instructions generated by the computer 454, for example. Under ideal situations, the illumination sources 470 output a predetermined intensity of light having a predetermined spectrum. When an illumination source encounters a fault, the light intensity and/or spectrum may change.

In the embodiment illustrated in FIG. 4, the quality check module 342 includes three imaging devices 452. Other modules and other embodiments of imaging modules may include fewer or more imaging devices. The imaging devices 452 may constitute one or more of the sensors 342A and image data generated by the imaging devices 452 may serve as sensor data for the AI algorithm 132. The imaging devices 452 capture images of the specimen container 102 and other objects in the imaging location. The imaging devices or processors associated therewith convert the images to image data that can be analyzed by the AI algorithm 132 as described herein.

The AI algorithm 132 may analyze image data generated by the imaging devices 452 to predict faults in components in the quality check module 342 and/or the diagnostic laboratory system 100 (FIG. 1). The image data also may be used by other algorithms to perform analysis of the specimen 210 (FIGS. 2A-2B). For example, the image data may be used to determine analyte concentrations within the specimen 210. In other embodiments, the image data may be used to determine whether the specimen 210 is in condition for analysis. For example, the analysis of the image data may determine whether at least one of hemolysis, icterus, and/or lipemia is present in the specimen. The analysis also may determine whether the specimen has a clot, a bubble, or foam, which may adversely impact future analysis. The image data and/or results of the analyses may be input to the AI algorithm 132 to predict faults and/or to train the AI algorithm 132.

In some embodiments, the image data may be used to identify the height HSP (FIG. 2), the height HGS, and/or the height HR. The heights may be used to calculate the volumes of the serum or plasma 210A (FIG. 2B) and/or the clot 210B (FIG. 2B). The volume information may be analyzed by the AI algorithm 132 to determine possible future faults of one or more modules. For example, a low height HSP of the serum or plasma 210A may be indicative of centrifuge modules not centrifuging specimens completely or a centrifuge device beginning to fail. The height HSP, the height HGS, and/or the height HR may be used individually or collectively to calculate the distance a probe or the like may extend into the specimen container 102 to aspirate the serum or plasma 210A. The height HSP, the height HGS, and/or the height HR may also be used as data to train the AI algorithm 132.

Referring again to FIG. 3, the instrument 318 may include other modules. In the embodiment illustrated in FIG. 3, the instrument 318 may include an aspiration and dispensing module 344 that may include one or more sensors 344A. Additional reference is made to FIG. 5, which illustrates a block diagram of an embodiment of the aspiration and dispensing module 344. Other embodiments of aspiration and/or dispensing modules may be used in the diagnostic laboratory system 100 (FIG. 1) and/or the instrument 318.

The aspiration and dispensing module 344 may aspirate and dispense specimens (e.g., specimen 210), reagents, and the like to enable the instrument 318 to perform chemical analyses, for example. The aspiration and dispensing module 344 may include a robot 550 that is configured to move a pipette assembly 552 within the aspiration and dispensing module 344. In the embodiment of FIG. 5, a probe 552A of the pipette assembly 552 is shown aspirating a reagent 554 from a reagent packet 556. The specimen container 102 is shown in FIG. 5 with the cap 208 (FIG. 2) removed such as by a decapping module (not shown). The pipette assembly 552 also may be configured to aspirate the serum or plasma 210A from the specimen container 102.

The reagent 554, other reagents, and a portion of the serum or plasma 210A may be dispensed into a reaction vessel, such as a cuvette 558. The cuvette 558 is shown as being rectangular in cross-section. However, the cuvette 558 may have other shapes depending on analyses that are to be performed. In some embodiments, the cuvette 558 may be configured to hold a few microliters of liquid. The cuvette 558 may be made of a material that passes light for photometric analysis as described herein. In some embodiments, the material may pass light having a spectrum (e.g., wavelengths) from 180 nm to 2000 nm, for example. It is noted that only a portion of the serum or plasma 210A may be dispensed into the cuvette 558 and other portions of the serum or plasma 210A may be dispensed into other cuvettes (not shown). In addition, other reagents may be dispensed into the cuvette 558.

Some components of the aspiration and dispensing module 344 may be electrically coupled to a computer 560. In the embodiment of FIG. 5, the computer 560 may include a processor 560A and memory 560B. Programs 560C may be stored in the memory 560B and executed on the processor 560A. The computer 560 may also include a position controller 560E and an aspiration/dispense controller 560D that may be controlled by programs, such as the programs 560C stored in the memory 560B. In some embodiments, the computer 560 and the components therein may be implemented in the instrument computer 339 (FIG. 3) and/or the laboratory computer 126 (FIG. 1). The position controller 560E and/or the aspiration/dispense controller 560D may be implemented in separate devices in some embodiments.

The programs 560C may include algorithms that control and/or monitor components within the aspiration and dispensing module 344, such as the position controller 560E and/or the aspiration/dispense controller 560D. As described herein, one or more of the components may include one or more sensors that may be monitored by one of the programs 560C. The sensors described in FIG. 5 collectively may be the sensors 344A (FIG. 3). In some embodiments, at least one of the programs 560C may include an artificial intelligence algorithm, such as the AI algorithm 132 (FIG. 3). In some embodiments, data generated by the sensors may be transmitted to the AI algorithm, which may predict faults in the aspiration and dispensing module 344 and/or other components in the diagnostic laboratory system 100 (FIG. 1).

The robot 550 may include one or more arms and motors that are configured to move the pipette assembly 552 within the aspiration and dispensing module 344. In the embodiment of FIG. 5, the robot 550 may include an arm 562 coupled between a first motor 564 and the pipette assembly 552. The first motor 564 may be electrically coupled to the computer 560 and may receive instructions from the position controller 560E. The instructions may instruct the first motor 564 as to direction and speed of the first motor 564. The first motor 564 may be configured to move the arm 562 to enable the probe 552A to aspirate and/or dispense specimens and/or reagents as described herein.

The first motor 564 may include or be associated a current sensor 566 that is configured to measure current drawn by the first motor 564. Data (e.g., measured current) generated by the current sensor 566 may be transferred to the computer 560. For example, the measured current may be data input to the AI algorithm 132 (FIG. 3). The AI algorithm 132 may use the measured current as an input to predict one or more faults in the aspiration and dispensing module 344 and/or the diagnostic laboratory system 100 (FIG. 1). The measured current may also be used to train the AI algorithm 132.

A second motor 568 may be coupled between the arm 562 and the pipette assembly 552 and may be configured to move the probe 552A in a vertical direction (e.g., a Z-direction) to aspirate and/or dispense as described herein. The second motor 568 may move the probe 552A in response to instructions generated by the programs 560C. For example, the second motor 568 may enable the probe 552A to descend into and retract from the specimen container 102, the cuvette 558, and/or the reagent packet 556. Liquids may then be aspirated and/or dispensed as described herein.

The second motor 568 may include or be associated a current sensor 570 that is configured to measure current drawn by the second motor 568. The data (e.g., measured current) generated by the current sensor 570 may be transferred to the computer 560. The measured current may be data input to the AI algorithm 132 (FIG. 3). The AI algorithm 132 may use the measured current as an input to predict one or more faults in the aspiration and dispensing module 344 and/or the diagnostic laboratory system 100 (FIG. 1). The measured current may also be used to train the AI algorithm 132.

The aspiration and dispensing module 344 may also include a vibration sensor 572 and a position sensor 574. In the embodiment of FIG. 5, the vibration sensor 572 and the position sensor 574 are mechanically coupled to the robot 550. In some embodiments, the vibration sensor 572 and/or the position sensor 574 may be coupled to other components in the aspiration and dispensing module 344. The aspiration and dispensing module 344 may include other vibration sensors and position sensors.

The vibration sensor 572 may be configured to measure vibration in the robot 550 and generate vibration data. The vibration data may be transmitted to the computer 560 and may ultimately be a data input to the AI algorithm 132 (FIG. 1) and used to predict faults in the aspiration and dispensing module 344 and/or the diagnostic laboratory system 100 (FIG. 1). For example, excessive vibration may be an indication of an impending fault in the robot 550 and/or other components. In some embodiments, the vibration data may be used to train the AI algorithm 132.

The position sensor 574 may be configured to sense positions of one or more components of the robot 550 or other components within the aspiration and dispensing module 344, such as the pipette assembly 552. In the embodiment of FIG. 5, the position sensor 574 may measure the position of the arm 562, the pipette assembly 552, and/or the probe 552A and may generate position data. The position data may be transmitted to the computer 560 and may ultimately be a data input to the AI algorithm 132 (FIG. 1) and used to predict faults in the aspiration and dispensing module 344 and/or the diagnostic laboratory system 100 (FIG. 1). For example, erratic position data may be indicative of moving components failing in the robot 550 and/or other components. In some embodiments, the position data may be used to train the AI algorithm 132.

The aspiration and dispensing module 344 may also include a pump 578 mechanically coupled to a conduit 580 and electrically coupled to the aspiration/dispense controller 560D. The pump 578 may generate a vacuum or negative pressure (e.g., aspiration pressure) in the conduit 580 to aspirate liquids. The pump 578 may generate a positive pressure (e.g., dispense pressure) in the conduit 580 to dispense liquids.

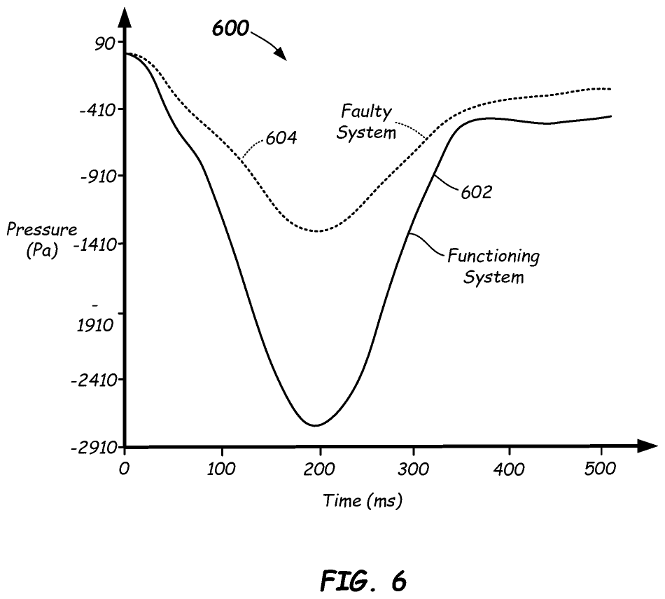

A pressure sensor 582 may measure the pressure in the conduit 580 and generate pressure data. In some embodiments, the pressure sensor 582 may be configured to measure aspiration pressure and generate pressure data. In some embodiments, the pressure sensor 582 may be configured to measure dispense pressure and generate pressure data. The pressure data may be in the form of a pressure trace as a function of time and as described with reference to FIG. 6 below. The pressure data may be transmitted to the computer 560 and may be used by the aspiration/dispense controller 560D to control the pump 578. The pressure data also may be input to the AI algorithm 132 (FIG. 1) to predict one or more impending faults in the pipette assembly 552 and/or the diagnostic laboratory system 100. The pressure data may also be used to train the AI algorithm 132.

Additional reference is made to FIG. 6, which is a graph 600 illustrating pressure traces of a pipette assembly aspirating a specimen showing a functioning aspiration/dispense system and a faulty aspiration/dispense system according to one or more embodiments. The aspiration/dispense system includes the pump 578, the conduit 580, and/or the pipette assembly 552. The pressure trace 602 illustrates a trace of a functioning aspiration/dispense system that shows a high vacuum during aspiration. The pressure trace 604 illustrates a trace of a faulty aspiration/dispense system. The pressure trace 604 shows a low vacuum, which may be indicative of a leak in the conduit 580, a weak pump, the pipette assembly 552 not entering the specimen fully, and/or other faults, which may be predicted by the AI algorithm 132 (FIG. 1). During a dispense operation, the faulty aspiration/dispense system will have low pressure. The pressure traces may be data input to the AI algorithm 132 (FIG. 1) to predict faults in the aspiration and dispensing module 344 and/or the diagnostic laboratory system 100. The pressure traces may also be used to train the AI algorithm 132.

Referring again to FIG. 5, the aspiration and dispensing module 344 also may include a temperature sensor 584 and/or a humidity sensor 586. The temperature sensor 584 may measure temperature, such as ambient temperature, in the aspiration and dispending module 344 and may generate temperature data. The humidity sensor 586 may measure humidity, such as ambient humidity, in the aspiration and dispensing module 344 and may generate humidity data. The temperature data and the humidity data may be transferred to the computer 560 and may ultimately be data input to the AI algorithm 132, which may predict one or more faults based at least in part on the temperature and/or humidity data. The temperature data and/or the humidity data may be used to train the AI algorithm 132. The ambient temperature and/or humidity may cause one or more faults to be premature. For example, when the aspiration and dispensing module 344 is operated in unnormal humidity and/or temperature, certain components within the aspiration and dispensing module 344 may experience premature faults, which may be predicted by the AI algorithm 132.

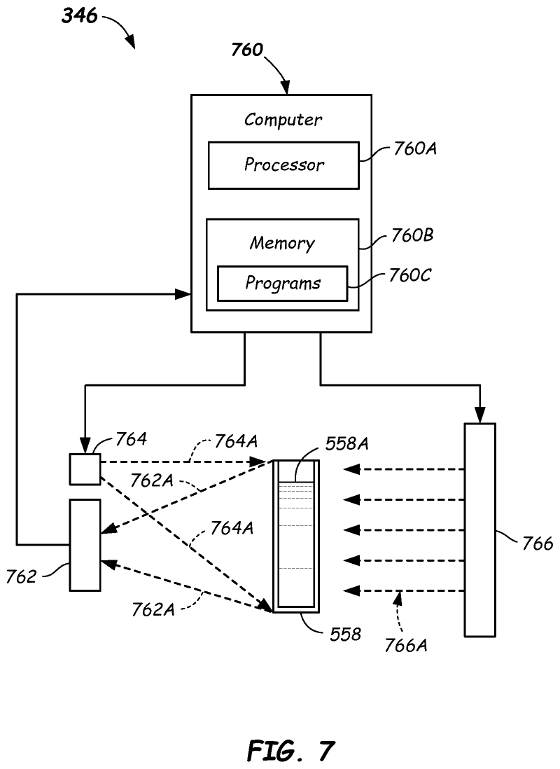

Additional reference is made to FIG. 7, which illustrates a block diagram depicting an embodiment of the analyzer module 346 (FIG. 3). The analyzer module 346 shown in FIG. 7 is an example of one of many different embodiments of analyzer modules that may be employed in the diagnostic laboratory system 100. The analyzer module 346 depicted in FIG. 7 performs analyses (e.g., photometric analyses) on the liquid 558A in the cuvette 558. The analyzer module 346 may include a computer 760 having a processor 760A and memory 760B that may store programs 760C executable by the processor 760A. Components of the analyzer module 346 may be controlled by programs 760C and data generated by the components may be analyzed and/or processed by the programs 760C.

The analyzer module 346 may include an imaging device 762 configured to capture one or more images of the liquid 558A and generate image data representative of the liquid 558A and/or light reflected by or passing through the liquid 558A. For example, the imaging device 762 may have a field of view 762A that enables the imaging device 762 to capture at least a portion of the liquid 558A when the cuvette 558 is located in an imaging location in the analyzer module 346. The image data may be processed by the programs 760C. The image data also may be data input to the AI algorithm 132 (FIG. 1) to predict faults in the analyzer module 346 and/or the diagnostic laboratory system 100 (FIG. 1).

The analyzer module 346 may include a front illumination source 764 and a back illumination source 766. The front illumination source 764 may be configured to emit light in a front illumination pattern 764A to illuminate the front of the cuvette 558 relative to the imaging device 762. The front illumination source 764 may be electrically coupled to the computer 760 and operated by instructions generated by the programs 760C executed by the processor 760A. In some embodiments, the intensity and spectrum of light emitted by the front illumination source 764 may be controlled by the programs 760C. Light reflected from the liquid 558A may be captured by the imaging device 762. Image data representative of the captured image may be analyzed by the computer 760, or another computer, to determine the presence and/or the concentration of one or more analytes in the liquid 558A. Other analyses also may be performed.

The back illumination source 766 may be configured to illuminate the back of the cuvette 558 relative to the imaging device 762 by a back illumination light pattern 766A. In some embodiments, the back illumination light pattern 766A may be substantially planar. For example, the back illumination source 766 may be a light panel. The back illumination light pattern 766A may provide for light emitted by the back illumination source 766 to pass through the liquid 558A. The back illumination source 766 may be electrically coupled to the computer 760 and operated by instructions generated by one or more programs 760C executed by the processor 760A. In some embodiments, the intensity and spectrum of light emitted by the back illumination source 766 may be controlled by the one or more programs 760C. The light passing through the liquid may be used by the imaging device 762 to capture an image of the liquid 558A. Image data representative of the captured image may be analyzed by the computer 760, or another computer, to determine the presence and/or the concentration of one or more analytes in the liquid 558A. Other analyses also may be performed.

In some embodiments, the imaging device 762, the imaging devices 452 (FIG. 4), and/or other imaging devices may be configured to measure light intensity. In some embodiments, the imaging device 762, the imaging devices 452, and/or other imaging devices may be configured to measure light frequency or light frequencies (e.g., spectrum or spectra). The light frequencies and/or the light intensities may be data inputs to the AI algorithm 132.

The image data generated by the imaging device 762 may be input to the AI algorithm 132 where it may be used to predict a fault in the analyzer module 346 or another component in the diagnostic laboratory system 100 (FIG. 1). The analysis of the liquid 558A may also be used by the AI algorithm 132 to predict a fault in the analyzer module 346 or another component in the diagnostic laboratory system 100. For example, if the analyses are consistently showing analyte concentrations rising, the AI algorithm 132 may determine that the rise is due to a component fault and not to concentrations in specimens continuously rising. Component faults may be, for example: a faulty front illumination source 764, a faulty back illumination source 766, a faulty imaging device 762, faulty centrifuging, and other components or processes in the diagnostic laboratory system 100 (FIG. 1).

Referring to FIGS. 1 and 3, the AI algorithm 132 is not a simple lookup table, but rather may include a model trained by supervised learning or unsupervised learning. Supervised learning includes a machine-learning task of learning a function that maps an input to an output based on example input-output pairs. Supervised learning infers a function from labeled training data consisting of a set of training examples. Labelled training data includes functions, such as known sensor data (e.g., sensor outputs) that are used as inputs to train the AI algorithm 132. In supervised learning, each example is a pair consisting of an input object, such as data from a sensor (typically a vector) and a desired output value (also called the supervisory signal). The output value may be a probability of a fault occurring within a predetermined period or a probability of a fault occurring at a specific time. The faults may be directed at specific components, instruments 118, and/or modules 120 in the diagnostic laboratory system 100.

The AI algorithm implemented as a supervised learning algorithm analyzes training data and produces an inferred function, which can be used for mapping new examples of faults. Lookup tables and the like do not produce at least the inferred function. The AI algorithm 132 may determine unforeseen fault scenarios, which cannot be accomplished using lookup table and the like. Accordingly, the AI algorithm 132 generalizes from the training data to predict unforeseen faults based on the training data and/or data generated by the sensors during operation of the diagnostic laboratory system 100.

Unsupervised learning may include AI algorithms that look for previously undetected patterns in data sets (e.g., sensor data) with no pre-existing labels and with a minimum amount of human supervision. In contrast to supervised learning that usually makes use of human-labeled data, unsupervised learning enables modeling of fault probabilities based on inputs, such as sensor data.

Two example processes used in unsupervised learning are principal component analysis and cluster analysis. Cluster analysis is used in unsupervised learning to group, or segment, datasets (e.g., sensor data) with shared attributes to extrapolate algorithmic relationships. Cluster analysis groups data (e.g., sensor data) that has not been labelled, classified, or categorized. Instead of responding to feedback, cluster analysis identifies commonalities in the data and predicts the faults based on the presence or absence of commonalities in each new sensor data. Cluster analysis enables the AI algorithm 132 to predict faults that may not fall into the commonalities.

The AI algorithm 132 may be implemented as a support vector machines, a linear regression, a logistic regression, a neural network, a generative network (e.g., a deep generative network, and other algorithms, for example. Training algorithms for the AI algorithm and/or the AI algorithm 132 may include, for example: vector machines, linear regression, logistic regression, naive Bayes, linear discriminant analysis, decision trees, k-nearest neighbor algorithms, neural networks (e.g., multilayer perceptron), recurrent neural networks, and similarity learning.

The AI algorithm 132 may be trained by user inputs correlated with various fault scenarios. For example, data from one or more of the sensors may be input into the AI algorithm 132 to generate a state of the diagnostic laboratory system 100 and/or instruments 118 or modules 120. Components that have experienced faults or that are experiencing faults may be input into the AI algorithm 132 to train the AI algorithm 132. In some embodiments, sensor measurements may be input to the AI algorithm 132 to generate a status of the diagnostic laboratory system 100, one or more of the instruments 118, and/or one or more of the modules 120.

Faults in the diagnostic laboratory system 100, the instruments 118, and/or the modules 120 may also be input into the AI algorithm 132. Faults input to the AI algorithm 132 may include one or more faulty instruments, one or more faulty modules, and/or one or more faulty components in the diagnostic laboratory system 100, the instruments 118, and/or the modules 120. For example, one or more motors or bearings prematurely failing may correspond to certain acoustic sensor data in combination with certain temperature data. These corresponding measurements may be subtle and may be identified by the AI algorithm 132. In other embodiments, certain pressure traces, such as the pressure trace 604 (FIG. 6A) may be indicative of a leak in the pipette assembly 552 (FIG. 5). In other examples, the AI algorithm 132 analyze the pressure trace 604 in combination with high current drawn measured by the pressure sensor 582 and may determine that the pump 578 is experiencing a fault or that a fault is imminent.

During operation of the diagnostic laboratory system 100, the sensor data may be periodically or continuously input to the AI algorithm 132. The data may be input into the AI algorithm 132 may be in the form of an array of values, wherein the values are data values from the sensors. The AI algorithm 132 may use the data to predict faults in instruments 118, modules 120, and/or other components in the diagnostic laboratory analyzer 100. The AI algorithm also may use the data to determine a status of the diagnostic laboratory analyzer 100, the instruments 118, and/or the modules 120. A technician servicing the diagnostic laboratory analyzer 100 may input the status of components into the AI algorithm 132, which may further train the AI algorithm 132. For example, the technician may indicate the status of bearing, motors, conduits, and other mechanical components. The technician also may indicate whether dirt is present on imaging devices, such as the imaging devices 452 (FIG. 4) or on the illumination sources, such as the illumination sources 470 (FIG. 4). The technician also may indicate whether any imaging components are out of alignment. The technician also may indicate the status of robots, such as the robot 550 (FIG. 5) and pipette assemblies, such as the pipette assembly 552. This data input by the technician in conjunction with the status of the diagnostic laboratory system 100 may further train the AI algorithm 132 to predict faults in the diagnostic laboratory system 100.

The AI algorithm 132 may make different fault predictions. These predications may be output to users of the diagnostic laboratory system 100, such as in the form of notifications and/or alerts. In some embodiments, the AI algorithm 132 may predict that there is a chance, such as a predetermined probability or risk score, that the diagnostic laboratory system 100 will experience a fault within a predetermined period of time. In response to the probability being greater than a predetermined value, the AI algorithm 132 may notify users of impending faults. For example, the AI algorithm 132 may predict that there is an 85% chance that the diagnostic laboratory system 100 will experience a fault within the next seven days.

In some embodiments, the AI algorithm 132 may predict a probability of fault to one or more components, instruments 118, and/or modules 120 in the diagnostic laboratory system 100. The probability of fault may be within a predetermined time period. For example, the AI algorithm 132 may determine that there is an 85% probability that the second instrument 118B will experience a fault in the next seven days. In some embodiments, the AI algorithm 132 may predict probabilities of faults within specific components of the instruments 118 and/or the modules 120.

In some embodiments, the AI algorithm 132 may predict probabilities as to when certain components will experience faults. For example, the AI algorithm 132 may predict when components have a greater than 85% chance of experiencing a fault. Thus, the AI algorithm 132 may generate a list indicating when certain components are likely to experience faults.

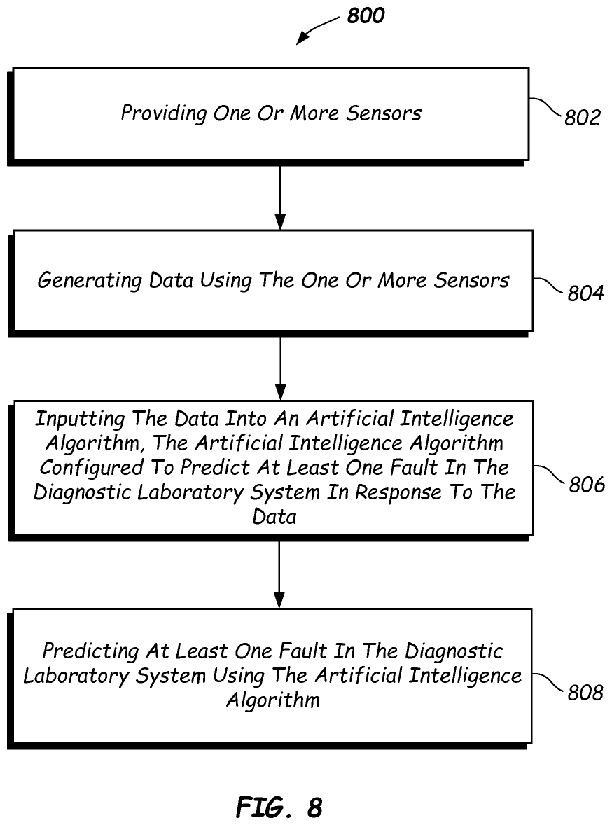

Reference is made to FIG. 8, which illustrates a flowchart depicting a method 800 of predicting a fault in a diagnostic laboratory system (e.g., diagnostic laboratory system 100). The method 800 includes, in 802, providing one or more sensors (e.g., sensors 338A, 340A, 342A, 344A, 336A). The method 800 includes, in 804, generating data using the one or more sensors. The method 800 includes, in 806 inputting the data into an artificial intelligence algorithm (e.g., artificial intelligence algorithm 132), the artificial intelligence algorithm configured to predict at least one fault in the diagnostic laboratory system in response to the data. The method includes, in 808, predicting at least one fault in the diagnostic laboratory system using the artificial intelligence algorithm.

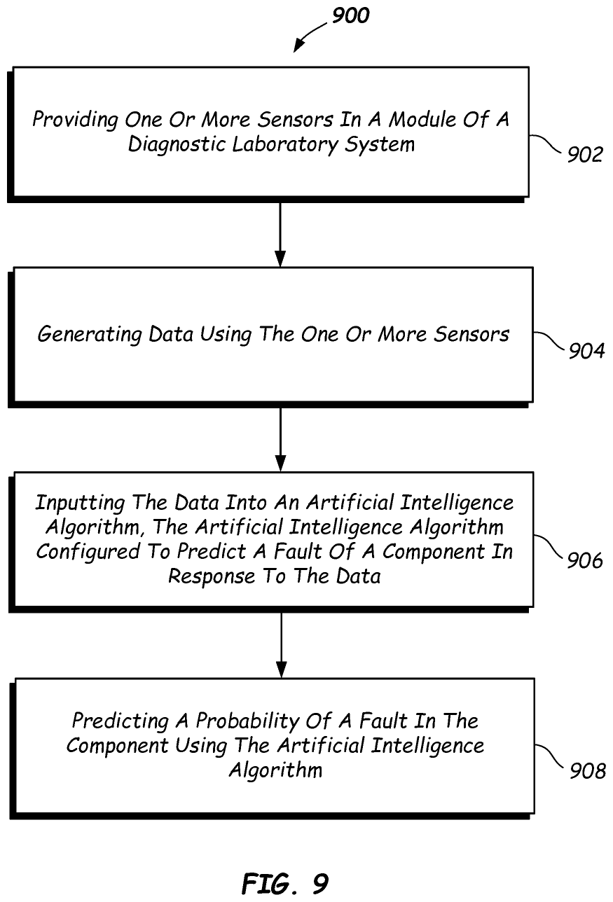

Reference is made to FIG. 9, which illustrates a flowchart depicting a method 900 of predicting a fault in a component of a module (e.g., one of modules 120, one or more modules 336) in a diagnostic laboratory system (e.g., diagnostic laboratory system 100). The method 900 includes, in 902, providing one or more sensors (e.g., sensors 338A, 340A, 342A, 344A, 336A) in the module of the diagnostic laboratory system. The method 900 includes, in 904, generating data using the one or more sensors. The method 900 includes, in 906, inputting the data into an artificial intelligence algorithm (e.g., artificial intelligence algorithm 132), the artificial intelligence algorithm configured to predict a fault of the component in response to the data. The method 900 includes, in 908, predicting a probability of a fault in the component using the artificial intelligence algorithm.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure but, to the contrary, to cover all modifications, equivalents, and alternatives falling within the scope of the claims.

What is claimed is:

1. A method of predicting a fault in a diagnostic laboratory system, comprising:
providing one or more sensors;
generating data using the one or more sensors;
inputting the data into an artificial intelligence algorithm, the artificial intelligence algorithm configured to predict at least one fault in the diagnostic laboratory system in response to the data; and
predicting at least one fault in the diagnostic laboratory system using the artificial intelligence algorithm.

2. The method of claim 1, wherein at least one of the one or more sensors is configured to measure aspiration pressure and wherein generating data comprises generating data indicative of aspiration pressure.

3. The method of claim 1, wherein at least one of the one or more sensors is configured to measure dispense pressure and wherein generating data comprises generating data indicative of dispense pressure.

4. The method of claim 1, wherein at least one of the one or more sensors is configured to measure electric current and wherein generating data comprises generating data indicative of electric current.

5. The method of claim 1, wherein at least one of the one or more sensors is configured to measure light intensity and wherein generating data comprises generating data indicative of light intensity.

6. The method of claim 1, wherein at least one of the one or more sensors is configured to measure light frequency and wherein generating data comprises generating data indicative of light frequency.

7. The method of claim 1, wherein at least one of the one or more sensors is configured to generate image data of a specimen and wherein generating data comprises generating image data of the specimen.

8. The method of claim 1, wherein at least one of the one or more sensors is configured to generate image data of a specimen container and wherein generating data comprises generating image data of the specimen container.

9. The method of claim 1, wherein at least one of the one or more sensors is configured to measure temperature and wherein generating data comprises generating data indicative of temperature.

10. The method of claim 1, wherein at least one of the one or more sensors is configured to measure humidity and wherein generating data comprises generating data indicative of humidity.

11. The method of claim 1, wherein at least one of the one or more sensors is configured to measure sound and wherein generating data comprises generating data indicative of sound.

12. The method of claim 1, wherein predicting comprises encoding data from the one or more sensors into an array of values indicative of a state of the diagnostic laboratory system, and wherein inputting the data comprises inputting the array of values into the artificial intelligence algorithm.

13. The method of claim 1, wherein the predicting comprises calculating a probability that a fault in the diagnostic laboratory system will occur within a predetermined period of time.

14. The method of claim 1, wherein the predicting comprises calculating a probability a fault of a module in the diagnostic laboratory system within a predetermined period of time.

15. The method of claim 14, comprising generating a notification in response to the probability being greater than a predetermined value.

16. The method of claim 1, wherein the predicting comprises predicting a probability that a component in a module of the diagnostic laboratory system will experience a fault within a predetermined period of time.

17. The method of claim 1, wherein the predicting comprises predicting a time when a module within the diagnostic laboratory system will experience a fault.

18. The method of claim 1, wherein the predicting comprises predicting a time when a component of a module within the diagnostic laboratory system will experience a fault.

19. The method of claim 1, comprising training the artificial intelligence algorithm.

20. The method of claim 1, wherein the artificial intelligence algorithm comprises a generative network.

21. A method of predicting a fault in a component of a module in a diagnostic laboratory system, comprising:
providing one or more sensors in the module of the diagnostic laboratory system;
generating data using the one or more sensors;
inputting the data into an artificial intelligence algorithm, the artificial intelligence algorithm configured to predict a fault of the component in response to the data; and
predicting a probability of a fault in the component using the artificial intelligence algorithm.

22. A diagnostic laboratory system, comprising:
one or more sensors configured to generate data; and
a computer configured to execute an artificial intelligence algorithm, the artificial intelligence algorithm configured to:
receive the data; and
predict at least one fault in a component of the diagnostic laboratory system in response to the data.
